(19) United States
(12) Patent Application Publication
(10) Pub. No.: US 2008/0255648 A1
(43) Pub. Date: Oct. 16, 2008

(54) Accessory for Muscle Maintenance and/or Muscle Development

(75) Inventors: Herve Coral, Le Landin (FR); Patrick Herbert, Bourguebus (FR)

Correspondence Address: CANTOR COLBURN, LLP, 20 Church Street, 22nd Floor, Hartford, CT, 06103, US

(73) Assignee: SPORT ELEC SA, Bourgtheroulde Infreville (FR)

(21) Appl. No.: 12/090,168

(22) PCT Filed: Oct. 14, 2005
(86) PCT No.: PCT/FR2005/002545

§ 371 Date: Apr. 14, 2008

(51) Int. Cl. A61N 1/22

(52) U.S. Cl. 607/149

(57) ABSTRACT

The invention concerns an accessory (2) designed to be positioned around a user's body so as to retain a plurality of electrode modules (19), arranged to be connected to at least one electric power generator for localized muscle maintenance and/or muscle development, characterized in that it is equipped with linking means (5) adapted to be removably connected to those provided by another accessory so as to form an accessory of larger dimensions.

### BACKGROUND OF THE INVENTION

The present invention relates to an accessory which is equipped with a plurality of electrodes and is intended to be positioned around a user's body in order to ensure localized muscle maintenance and/or muscle development.

### DESCRIPTION OF THE PRIOR ART

Such an accessory is conventionally intended to cover a plurality of electrode modules placed in contact with the user's skin. More precisely these electrode modules are adapted to domestic-use generators, and they generally consist of a flexible conductive element whose face, intended to come in contact with the skin, is covered with a layer made of a material that allows both the passage of an electrical current and adhesion to the skin.

Depending on whether the muscles to be trained are the abdominal wall muscles, the biceps, the pectorals, or the quadriceps and the hip-leg muscles, for example, the accessory used will have the form of a belt, an armband, a vest or a leg-strap.

The user will therefore necessarily have to obtain each of these various accessories if they wish to work the main muscles of their body, which clearly constitutes a significant drawback in terms of cost and encumbrance.

### SUMMARY OF THE INVENTION

It is an object of the present invention to remedy the problem mentioned above, and to that end it provides an accessory intended to be positioned around a user's body in order to hold a plurality of electrode modules, which are intended to be connected to at least one electrical energy generator and which permit localized muscle maintenance and/or muscle development, characterized in that it is equipped with attachment means adapted to be connected removably to those placed on another accessory, with a view to forming an accessory having larger dimensions.

An accessory according to the invention will thus advantageously have the dimensions of a conventional armband or a conventional leg-strap, and the temporary connection of two accessories according to the invention will make it possible to form an accessory with larger dimensions, for example a belt. It is therefore possible to train the main parts of the body with just two accessories, which are used separately or connected to one another, as appropriate.

According to a first preferred embodiment of the invention the attachment means comprise at least one strip of looped fabric, or as a variant a strip of a hooked fabric. In this case, the removable fastening of the accessories to one another is fastening of the Velcro® type.

According to a second preferred embodiment of the invention, the attachment means consist of at least one hook. As a variant, one of the two accessories may have at least one hook and the other accessory may have at least one eyelet, corresponding with each hook.

According to a third preferred embodiment of the invention, the attachment means may consist of a zipper.

More generally, it should be understood clearly that the attachment means of an accessory according to the invention may consist of any device that allows rapid and easy assembly or separation of two accessories to be carried out.

An accessory according to the invention is preferably produced in the form of a band having a first end equipped with said attachment means.

Also preferably, the band has one face covered with at least one strip of hooked fabric in the vicinity of its second end. Complementarily, the other face of the band is preferably covered at least partially with at least one strip of looped fabric. Since the desired fastening is fastening of the Velcro® type, it is clear that as a variant the second end may be equipped with at least one strip of looped fabric and the other face of the band may be covered at least partially with at least one strip of hooked fabric.

A traditional drawback encountered when using an accessory is that the phase during which the electrode modules are put in place remains intricate and laborious. This is because each electrode module has to be dealt with individually, and the application of an intermediate layer used as a connection between the skin and the electrode module must be carried out perfectly in order to avoid any loss of adhesion and in order to ensure correct transmission of the electrical energy. This intermediate layer generally consists of a spongy fabric or an echography gel. In order to overcome this drawback, an accessory according to the invention advantageously has an inside face equipped with a plurality of protruding electrode modules, each electrode module specifically having a thickness increased owing to the incorporation of a lining pad made of a material which can be deformed in order to adapt optimally to the user's morphology. The electrode modules are consequently put in place very rapidly and without difficulty, and the contact between each electrode module and the user's skin is perfectly optimized even in the event of movements by the user. It is furthermore possible to replace the spongy fabric or the echography gel traditionally employed by a simple layer of a cosmetic cream.

In the context of the present invention, it should be understood clearly that the term “skin” corresponds equally well to the outer layer of the human body and to that of animals.

### DESCRIPTION OF THE DRAWINGS

The invention will be understood more clearly with the aid of the detailed description which is explained below with reference to the appended drawing, in which:

FIG. 1 is a perspective view of a belt comprising two accessories according to the first preferred embodiment of the invention;

FIG. 2 is an enlarged partial perspective view of the belt represented in FIG. 1, in the region of the attachment means;

FIG. 3 is a partial perspective view of another belt comprising two accessories according to the second preferred embodiment of the invention;

FIG. 4 is an exploded perspective view of an electrode module with which an accessory according to the invention is equipped;

FIG. 5 is a cross-sectional view of the electrode module represented in FIG. 4, once assembled.

### DESCRIPTION OF THE PREFERRED EMBODIMENTS

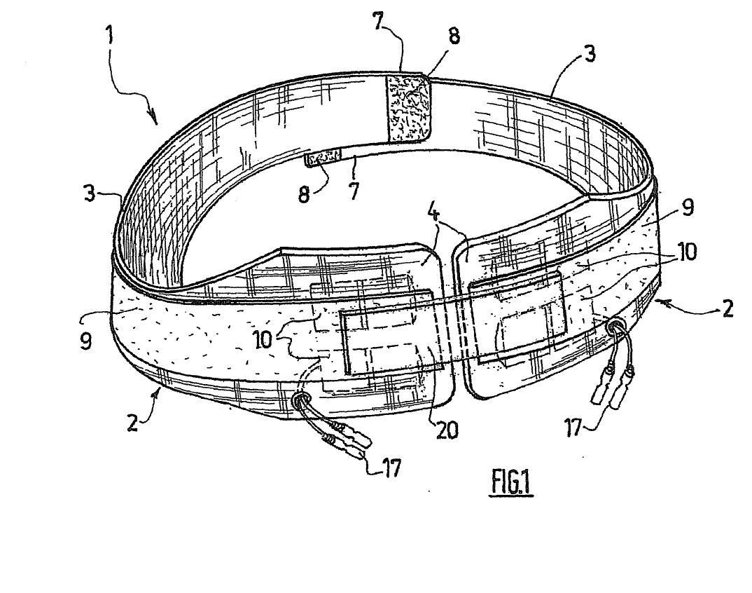

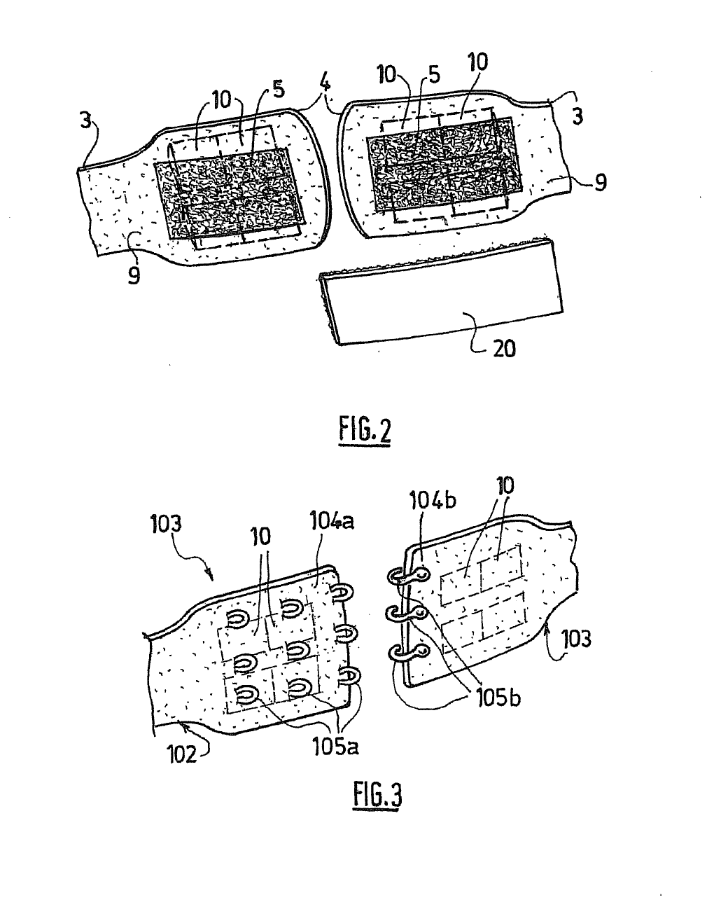

A belt 1 according to the invention, as represented in FIGS. 1 and 2, is obtained from two separate accessories 2 according to the first preferred embodiment of the invention.

More precisely, each accessory 2 is produced substantially in the form of a band 3 having a first end 4 provided with attachment means 5 on its outwardly oriented face. These attachment means 5 consist of a strip of looped fabric extending longitudinally in the central part of said band 3.

The band 3 furthermore has on the one hand an inside face covered strip of hooked fabric 8 in the region of its second end 7, and on the other hand an outside surface almost entirely covered with a strip of looped fabric 9.

The inside face of the band 3 is also equipped with the electrode modules 10.

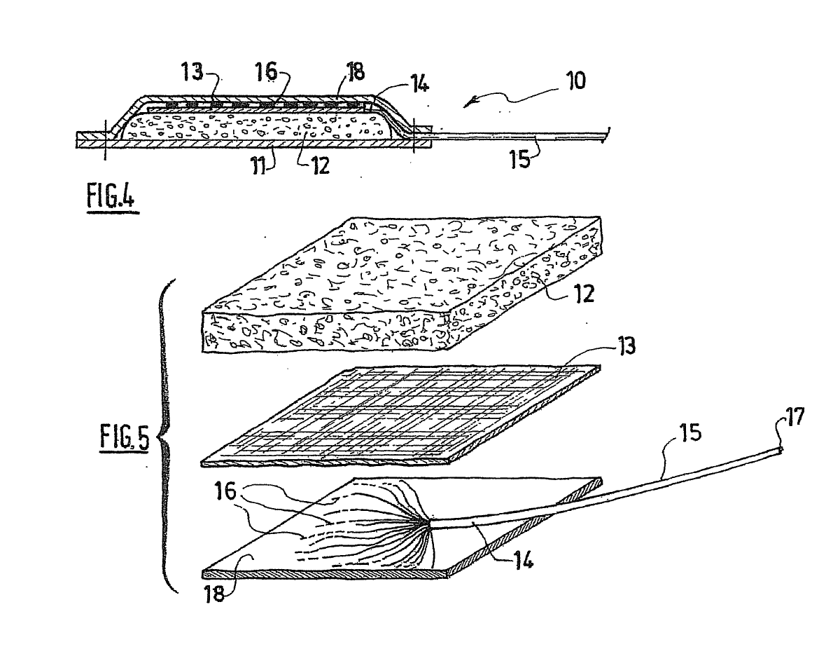

More precisely each electrode module 10, as represented in FIGS. 4 and 5, is obtained from an outer wall 11 produced in the form of a reinforced fabric plate (not shown in FIG. 4) with a substantially rectangular shape, made of a material such as 600 D polyester.

A lining pad 12, consisting of a parallelepipedal pad of polyurethane foam, is subsequently fitted on the outer wall 11.

A double-sided adhesive wall 13 with a rectangular shape is subsequently applied onto the lining pad 12, and adheres to it by one of its two faces.

A conductive end 14 of an electrical cable 15 is then applied onto the other face of the adhesive wall 13. More precisely, the conductive end 14 has the form of conductive filaments 16 arranged in a star-shape and adhesively bonded flatly onto the adhesive wall 13, and the electrical cable 15 is provided with an opposite second end 17 which is intended to be connected to an electrical energy generator (not shown).

Lastly, an inner wall 18 produced in the form of a substantially rectangular silicone-carbon plate is fitted onto the adhesive wall 13 and adheres to it. This inner wall 18 furthermore has a border 19 fixed, for example by welding, into the border which is on the outer wall 2.

Each electrode module 10 formed in this way is then ready to be fixed either temporarily for example by Velcro® type fastening, or permanently for example after a stitching operation, into the inside face of the band 3 in the region of its first end 4.

When the user wishes to engage in a muscle maintenance and/or muscle development session for one of their limbs, for example a thigh, the user starts by applying a layer of cosmetic cream then takes an accessory 2 and unrolls it around said limb. They then only need to fold the second end 7 onto the outside face of the band 3 in order to carry out Velcro® type fastening. The electrode modules 10 forming bulges then come perfectly in contact with the muscles that need to be stimulated, which makes it possible to optimize passage of the electrical current.

When the user wishes to engage in a muscle maintenance and/or muscle development session for their abdominal wall, they take two accessories 2 and place their first ends 4 against one another. They then take a thin piece of hooked fabric 20 and place it onto the two strips of looped fiber lying on the first ends 2 and constituting the attachment means 5. Once this has been done, the two accessories 2 are connected to each other and are converted into a new accessory with larger dimensions corresponding to said belt 1. The user then applies a layer of cosmetic cream onto a part of their abdominal wall, and unrolls the belt 1 around their waist. They then only need to fold the second end 4 of one of the two strips 3 onto the outside face of the band 3 in order to carry out Velcro® type fastening. As before, the electrode modules 10 forming bulges then come perfectly in contact with the muscles that need to be stimulated, in this case the muscles of the abdominal wall, which makes it possible to optimize passage of the electrical current.

Another belt 101 according to the invention, represented partially in FIG. 3, is obtained from two accessories 102, 103 according to the variant of the second preferred embodiment of the invention.

The accessories 102, 103 differ from the accessories 2 in that the attachment means of the accessory 102 now consist of three rows of eyelets 105a, which are fixed in the first end 104a and are adapted to engage with a row of hooks 105b constituting the attachment means with which the first end 104b of the accessory 103 is equipped.

More generally it should be understood clearly that the attachment means, with which an accessory according to the invention is equipped, may consist of any device that allows rapid and easy assembly or separation of two accessories to be carried out.

Although the invention has been described in connection with particular embodiments, it is very clear that it is not limited in any way thereto and that it comprises all the technical equivalents of the described means as well as combinations of them, so long as they fall within the scope of the invention.

1. An accessory (2; 102, 103) intended to be positioned around a user's body in order to hold a plurality of electrode modules (10), which are intended to be connected to at least one electrical energy generator and which permit localized muscle maintenance and/or muscle development, characterized in that it is produced in the form of a band (3) having a first end (4; 104a, 104b) equipped with said plurality of the electrodes, said first end being equipped with attachment means (5; 105a, 105b) adapted to be connected removably to those of another accessory in front of them, with a view to forming an accessory having larger dimensions.

2. The accessory (2) as claimed in claim 1, characterized in that the attachment means (5) comprise at least one strip of looped fabric.

3. The accessory (102) as claimed in claim 1, characterized in that the attachment means (105b) consist of at least one hook.

4. The accessory (2; 102, 103) as claimed in claim 1, characterized in that the band (3) has one face covered with at least one strip of hooked fabric (8) in the vicinity of its second end (7).

5. The accessory (2; 102, 103) as claimed in claim 4, characterized in that the other face of the band (3) is at least partially covered with at least one strip of looped fabric (9).

6. The accessory (2; 102, 103) as claimed in claim 1, characterized in that it has an inside face equipped with a plurality of protruding electrode modules (10).

7. A belt (1; 101), characterized in that it consists of two accessories (2; 102, 103) as claimed in claim 1 connected to one another.
